(19) United States
(12) Patent Application Publication
(10) Pub. No.: US 2006/0122494 A1
(43) Pub. Date: Jun. 8, 2006

(54) Method and system for localising an ingestible element for the functional investigation of the digestive tract

(76) Inventor: Michel Luc Bouchoucha, Paris (FR)

Correspondence Address: GREER, BURNS & CRAIN, 300 S WACKER DR, 25TH FLOOR, CHICAGO, IL, 60606, US

(21) Appl. No.: 10/522,055

(22) PCT Filed: Jul. 22, 2003
(86) PCT No.: PCT/FR03/02314

§ 371 Date: Aug. 8, 2005

(30) Foreign Application Priority Data
Jul. 25, 2002 (FR) 02/09420

(51) Int. Cl. A61B 5/05

(52) U.S. Cl. 600/424

(57) ABSTRACT

A method for non-invasive ambulatory exploration, includes causing the subject to swallow at least one emitting element ingestible but non-digestible including a device for transmitting at a given fixed frequency; measuring, at a given time using at least three reception devices distributed around the subject's trunk, the phase shift of the frequency transmitted by the transmission devices relative to a reference phase; determining by triangulation on the basis of the three phase-shift measurements the position of the element; defining based on the position of the element a digestive motility and/or passage data. Thus, it is possible to assess: gastric emptying time, gut and colic motility; depending on the weight of the transmitter, gastric, intestinal and colic activity and propulsive force of the stomach, the bowel and the colon; the length of the small intestine and of the colon.

The present invention relates to a method of non-invasive ambulatory exploration allowing the assessment of digestive motricity and/or transit, and a corresponding system.

Currently the techniques proposed for measuring the digestive motricity are invasive, requiring intubation of the patient (manometric and electromyographic techniques).

In order to measure the transit, isotropic or radiopaque markers are used.

These known techniques in particular have the following drawbacks:
they cannot be easily applied in everyday clinical practice and require a very significant human and material input;
problems of toxicity are encountered in particular in children, pregnant women;
it is often impossible to correlate the phenomena recorded for studying the motricity of an organ with transit measurements.

The purpose of the present invention is to overcome these drawbacks and to propose a method and a system of non-invasive exploration which can, at a low cost, assess the function of each organ involved in digestion (stomach, small intestine, colon), and more particularly simultaneously assess the digestive motricity and transit.

It proposes a method of non-invasive exploration for assessing the digestive motricity and/or transit of a human or animal subject, characterized in that it consists of:
said subject swallowing an ingestible transmitting element which is non-digestible containing means transmitting at a given fixed frequency;
measuring, at a given time using at least three frequency reception means distributed around said subject's trunk, the phase shift of the frequency transmitted by said transmission means relative to a reference phase;
determining by triangulation on the basis of the three phase-shift measurements the position of said element;
defining, according to the position of said element, a data for the assessment of the digestive motricity and/or transit.

The present invention also proposes a corresponding system, characterized by:
on the one hand:
   an ingestible transmitting element which cannot be digested by said subject containing means transmitting at a given fixed frequency; and
on the other hand:
   frequency receiving means comprising at least three frequency receivers intended to be placed around the trunk of said subject, each receiver being able to measure at a given time the phase shift of said transmission frequency relative to a reference phase;
   means for processing and analyzing the three phase-shift measurements made by said receivers which are able to determine, by triangulation, the position of said element.

Thus, according to the invention, a transmitter circulates in the digestive system, while the data collection takes place in an ambulatory manner using at least three receivers distributed for example on the circumference of the abdominal belt. The basic principle is to measure at a given time the phase shift which is produced between two identical frequencies when the distance from the transmitting source relative to its reference position (corresponding for example to the transmitter in the mouth) is varied. Preferably a high-frequency transmission is chosen because it allows improvement in the precision of the measurements. Using three phase-shift measurements made approximately simultaneously, it is then possible to determine, by triangulation, the position in a three-dimensional reference frame of the transmitter and to deduce a characteristic relating to the digestive motricity and/or transit (for example using software including data interpretation).

The solution proposed by the invention is:

ambulatory and no longer invasive: only the transmitting element is ingested, but in the same way as a capsule of medicinal product or similar; the receivers are worn by the subject, for example mounted on a standard belt; the patient is free in his movements, his relative position having no influence;

non-toxic: materials exist which can constitute an envelope for the transmitter which are suitable in terms of consistency and non-toxic, for example plastic materials (non-digestible); the transmitting element is for single-use and is eliminated naturally (the risk of cross infection is eliminated);

it allows the assessment of the function of an organ involved in digestion: by monitoring the position of the transmitting element, it is possible to measure:

gastric emptying time;

intestinal transit time;

colon transit time;

knowing the weight of the transmitting element, it is possible to measure:

gastric activity

intestinal activity;

colic activity;

the propulsive force of the stomach, the intestine and the colon.

low cost of the technique of the invention: the manufacturing costs of the reception element are low: standard miniature electronic components can be used in an impervious envelope; the equipment required by the doctor is limited (belt and analysis software); moreover, in a single examination information is obtained which previously was obtained by different explorations; it should be noted here that the invention could meet, in a simple and inexpensive way, a real requirement of doctors: the number of diagnoses of constipation/diarrhoea was estimated in 1990 at 300,000 by 500 gastroenterologists surveyed (Dorema figures).

Furthermore, information which no other examination could previously give can be provided by the system according to the invention:

measurement of the length of the small intestine; and

measurement of the length of the colon.

According to other characteristics of the invention, the number of frequency receivers can be greater than three in order to refine the triangulation and increase the precision of the system and also in order to be able to rule out a measurement which is clearly wrong. Moreover, memory means, which can be common to the receivers, will be used to store in an ambulatory manner the measurements made by the frequency receivers, the analysis and processing of the data then being able to be carried out by subsequently connecting these memory means to a central processing unit, for example a personal computer, equipped with appropriate analysis and processing software. Furthermore, the transmitting element can comprise an integrated and/or induced power supply with, for example, a standby mode and an active mode. In the case of an induced power supply, induction means can be placed on the abdominal circumference, for example on the same belt as that carrying the receivers. Means can also be provided for controlling the transmission at time intervals chosen for the measurements, allowing an appropriate monitoring of the transmitting element.

The present invention will be better understood and other advantages and characteristics will become apparent in light of the following description of an embodiment, which refers to the drawings in which:

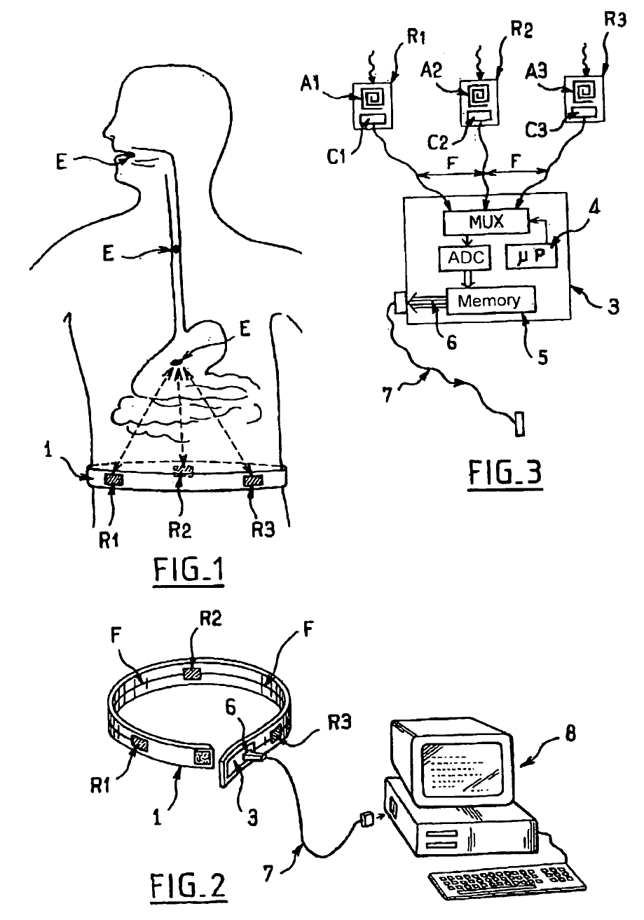

FIG. 1 is a schematic view of an embodiment of the system according to the invention, showing a man wearing a belt which is part of the system and having ingested the transmitting element;

FIG. 2 illustrates a step of recovering and processing the data recorded in an ambulatory manner; and

FIG. 3 schematically illustrates an embodiment of electronic components for processing the signals picked up.

According to the example chosen and represented in the figures, the system comprises a belt 1, with standard fastening means 2, on which three frequency receivers R1, R2 and R3 are arranged, each distributed at a distance from one another on the belt, at approximately the same distance. Each receiver, respectively R1, R2, R3, is constituted by a receiving antenna, respectively A1, A2 and A3, which is able to pick up a given high-frequency transmitted by a transmitting element E intended to be ingested by the subject. In each receiver R1, R2, R3, an electric circuit is provided which is able to convert the signal picked up by the antenna A1, A2, A3, into an electric signal and comprising a local amplifier. The three receivers R1, R2 and R3 are connected by cable way (symbolized by F in FIGS. 2 and 3) to a buffer card 3 also arranged on the belt 1 (see FIGS. 2 and 3). The card 3 comprises means for supplying energy 4, an MUX multiplexer, an analogue-to-digital converter ADC and memory means 5 which are able to store the measurements made by the three receivers R1, R2 and R3 at a given time or given time intervals and an output 6 for a connection cable 7, for example, of USB type. The cable 7 serves to transfer the digital data stored in the card 3 to data analysis and processing means, for example, a personal computer 8 equipped with appropriate software for interpretation of the data transmitted, for display, etc.

According to the invention, the system comprises the transmitting element E, represented in three positions in the human digestive system shown schematically in FIG. 1. Element E comprises means for transmitting at a given frequency. This can be an oscillator driven by a resonator, the output occurring on an antenna for the transmission. The transmitting element E also comprises integrated means for supplying transmitting means with energy, for example a metal oxide type battery and means for controlling said power supply means which allow the change from a standby mode to an active mode. The transmission frequency is preferably chosen to be high, for example 868 MHz, in order to increase the precision of the measurements, as was explained above. It is also chosen among the frequencies permitted in the medical field. The dimensions of the components must be compatible with the fact that the transmitting element is to be ingested. Electronic components of the standard SMC miniature components type can be used. Moreover, these different components are placed in an impervious envelope constituted by a non-digestible material, sufficiently rigid to pass in particular the pylorus at the exit of the stomach, which is non-toxic and non-allergenic. This can be for example an envelope made of plastic material in the form of a gelatin capsule, capsule or similar.

Moreover it is possible to use this system to measure physiological values such as pH, pressure, temperature, etc. For this purpose, the transmitting element will include a corresponding sensor. The transmission of measurements made by this sensor will be transmitted to the receiving means in the form of a modulation of the amplitude of the frequency transmitted by the transmitter corresponding to the amplitude of the signal provided by said sensor.

The system operates in the following way. The belt 1 is fitted around the waist of the subject. The transmitting element is placed in the mouth of the subject and a reference phase is determined and recorded in the card 3 at this location for the transmission frequency of the transmitter. The transmitting element E then moves naturally in the digestive system, while the subject is free in his movements. At certain selected time intervals, corresponding to a change in the power supply of the transmitting means from the standby mode to the active mode, the receivers R1, R2 and R3, simultaneously pick up the frequency transmitted by the element E. Each signal picked up is collected and stored in the memory card 3. When the examination is considered to be complete, the belt is removed from the subject and the card 3 can be connected at the desired time via the cable 7 to the computer 8, where the digital data from the card 3 will be able to be analyzed by the data analysis and processing software. For each trio of values of signals picked up, the software will determine the phase shift relative to the reference position, then it will determine by triangulation the 3D position of the receiving element and, using programmed interpretation instructions, it will provide results regarding the transit time in an organ, a distance covered, a length of a segment of the digestive system and as a function of the weight of the transmitting element it will provide results on the activity of an organ, for example its propulsive force. The transmitting element E is for single-use, and is eliminated naturally.

In order to allow a better quantization of the transit time of an organ, several transmitting elements can be ingested successively, for example on successive days, in order to produce a multiple measurement in a stable physiological state.

Of course other variant embodiments of the system are possible, in particular, means can be provided for programming the transmission of the transmitting element at selected intervals.

1. A method of non-invasive exploration for assessing the digestive motricity and/or transit of a human or animal subject, comprising:
said subject swallowing an ingestible transmitting element which is non-digestible containing means transmitting at a given fixed frequency;
measuring, at a given time using at least three reception means distributed around said subject's trunk, the phase shift of the frequency transmitted by said transmission means relative to a reference phase;
determining by triangulation on the basis of the three phase-shift measurements the position of said element;
defining, according to the position of said element, a data for the assessment of the digestive motricity and/or transit.

2. The method according to claim 1, characterized in that the measurements corresponding to the phase shift are stored in memory means.

3. The method according to claim 1, characterized in that the receiving means are placed around the abdominal belt.

4. The method according to claim 1, characterized in that a series of position measurements are made which are spread over time.

5. The method according to claim 1, characterized in that a position reference measurement is made when the element is in the mouth of the subject, before he swallows it.

6. The method according to claim 1, characterized in that the power supply of the transmitting element is triggered at given times and the corresponding phase-shift measurements at each given time are stored in the memory means.

7. The method according to claim 1, characterized in that the amplitude of the transmission frequency of the transmission means is modulated as a function of the amplitude of a signal picked up by a sensor included in the transmitting element, said sensor being able to pick up a signal representing a physiological characteristic.

8. The method according to claim 1, characterized in that said subject ingests several transmitting elements over a period of time, each transmitting element having a characteristic frequency.

9. A non-invasive exploration system for assessing the digestive motricity and/or transit of a human or animal subject, in particular for the implementation of the method according to claim 1, characterized by: on the one hand:
an ingestible transmitting elements which cannot be digested by said subject containing means transmitting at a given fixed frequency; and on the other hand:
receiving means comprising at least three receivers intended to be placed around the trunk of said subject, each receiver being able to measure at a given time the phase shift of said transmission frequency relative to a reference phase;
means for processing and analyzing the three phase-shift measurements made by said receivers which are able to determine, by triangulation, the position of said element.

10. The system according to claim 9, characterized in that it also comprises means for storing in the memory the phase-shift measurements made by the receivers at a given time.

11. The system according to claim 9, characterized by a high transmission frequency.

12. The system according to claim 10, characterized in that the transmitting elements comprises integrated power supply means.

13. The system according to claim 9, characterized in that the transmitting element comprises induced power supply means.

14. The system according to claim 9, characterized in that the receivers are distributed on a belt which is able to be fixed on the trunk of the subject.

15. The system according to claim 14, characterized in that the belt also comprises means for the induction of the power supply of said transmitting element.

16. The system according to claim 14, characterized in that the analysis and processing means include a card comprising means for analogue-to-digital conversion of the signals picked up and memory means common to the three receivers and arranged on the belt.

17. The system according to claim 9, characterized by means for connecting the memory means to the processing and analysis means and for transferring the data relating to the phase shifts measured.

18. The system according to claim 9, characterized in that the transmitting element comprises a sensor which is able to pick up a signal representing a physiological characteristic, the amplitude of the frequency transmitted by the transmission means being able to be modulated as a function of the amplitude of the signal picked up by said sensor.

19. The system according to claim 9, characterized in that it comprises several transmitting elements intended to be ingested by said subject over a period of time.
